(19) United States
(12) Patent Application Publication
(10) Pub. No.: US 2021/0347978 A1
(43) Pub. Date: Nov. 11, 2021

(54) PEROXIDE STABLE POLYMER COMPOSITION AND PROCESS FOR ITS PREPARATION AND APPLICATIONS THEREOF

(71) Applicant: ISP INVESTMENTS LLC, Wilmington, DE (US)

(72) Inventors: Thomas DURIG, Chadds Ford, PA (US); Paul C. GILLETTE, Newark, DE (US); Michael A. TALLON, Aberdeen, NJ (US)

(73) Assignee: ISP INVESTMENTS LLC, Wilmington, DE (US)

(21) Appl. No.: 17/277,686

(22) PCT Filed: Sep. 17, 2019
(86) PCT No.: PCT/US19/51546

§ 371 Date: Mar. 18, 2021

Related U.S. Application Data
(60) Provisional application No. 62/733,445, filed on Sep. 19, 2018.

(51) Int. Cl. C08L 39/06; C08L 31/04; A61K 45/06; A61K 47/32; A61K 47/10

(52) U.S. Cl. CPC C08L 39/06; C08L 31/04; C08K 5/06; A61K 47/32; A61K 47/10; A61K 45/06

(57) ABSTRACT

A peroxide stable polymer composition comprises a mixture of polyvinylpyrrolidone/vinyl acetate copolymer (PVP/VA) and butylated hydroxy anisole (BHA), an antioxidant. Products or applications comprising said stable polymer composition and a process for the preparation thereof are disclosed in the present application.

### FIELD OF THE INVENTION

The present invention relates to peroxide stable polymer compositions and applications thereof. More particularly, the present invention relates to a process for preparing peroxide stable polymer compositions comprising polyvinylpyrrolidone/vinyl acetate (PVP/VA) copolymer and butylated hydroxy anisole (BHA).

### BACKGROUND OF THE INVENTION

Polyvinylpyrrolidone (PVP) polymers are used in a wide range of industrial applications such as in pharmaceutical formulations as a binder, in adhesives to improve strength, in papers manufacture to increase strength, in synthetic fibers to improve dye receptivity and in inks/coatings. This can be attributed to its unique physical and chemical properties such as excellent solubility in both water and organic solvent system, non-toxic nature, and its affinity to complex with both hydrophilic and hydrophobic substances. However, PVP polymers are also known to be very susceptible to oxidative degradation caused by reactive peroxides present therein as impurity. The peroxides are detrimental to the PVP polymers as well as for the products derived therefrom as the presence of peroxides above threshold concentration negatively impacts polymer stability and performance.

PVP polymers are widely used as an excipient in pharmaceutical applications. Excipients play a very crucial part in the formulation of pharmaceutical dosages forms due to their significant contribution to the overall properties of the dosage forms. The presence of reactive peroxides can lead to degradation of oxidation-labile drugs along with their color degradation. Maintaining the peroxide level below threshold concentration is, therefore, an utmost concern. As per the current pharmacopeia, Ph. Eur. 6 and JP XIV, the peroxide content for these polymers is limited to a maximum of 400 ppm.

One of the primary sources of peroxides in PVP polymers is believed to be the use of peroxides to initiate the polymerization reaction. Some studies have also suggested that the introduction of peroxides may occur after synthesis during the drying process. The content of peroxides tends to increase further upon subsequent storage, packaging and handling.

A number of preventive measures have been adopted in the prior-art for controlling peroxide formation in PVP polymers. Initially, control of initial peroxide concentration was recommended. Other approaches employed for reducing peroxide content in PVP polymers include the use of enzymes, metals, additives, chemical modification of crosslinkers, supercritical fluid extraction, and vacuum drying. However, the peroxide impurities in PVP polymers tend to increase upon storage and remain problematic.

U.S. Pat. No. 8,623,978 discloses a process for the preparation of low-peroxide, water insoluble crosslinked vinyl lactam polymer (PVPP) by free-radical polymerization. In the process disclosed therein, the vinyl lactam based monomers are polymerized in the presence of antioxidants such as tocopherols, catechin hydrate, uric acid, propyl 3, 4, 5-trihydroxybenzoate, 4-hydroxy-2,2,6,6-tetramethylpiperidin-1-oxyl, tris(tetramethyl-hydroxypiperidinol) citrate, N-acetylcysteine, bis-(2,2,6,6-tetramethylpiperidin-1-oxyl-4-yl)decanedioate and 1,2-diothiolane-3-pentanoic acid, and crosslinkers.

United States Patent Application Publication No. 2011/0257339 discloses a process for preparing low-peroxide polymers such as polyamide, polyether, polyvinylamide (crosslinked water insoluble PVPP) in which the polymers are treated with elemental metals such as sodium, potassium, magnesium, calcium, zinc, platinum, palladium, rhodium, iridium, ruthenium, nickel, gold, or an alloy of these metals, in the presence of a liquid such as water.

U.S. Pat. No. 8,524,827 discloses a method for stabilizing polyvinylpyrrolidones in which the polyvinylpyrrolidones are treated with sulfur containing compounds such as sulfur dioxide, sulfurous acid or an alkali metal sulfite followed with free radical scavengers. The free radical scavengers as disclosed in this patent are ascorbic acid, nordihydroguaiaretic acid, ethoxyquin, bisabolol, asorbylpalmitate and BHT (“butylated hydroxytoluene”: 2,6-di-tert-butyl-4-methylphenol).

PCT Publication No. 2006083950 discloses a method for reducing the level of peroxides in biocompatible polymers by adding methionine to the polymer preparation. The biocompatible polymers disclosed in this PCT publication are polyvinylpyrrolidone, polyethylene glycol, or methyl cellulose.

In view of the foregoing, there still exists a need to provide polymer compositions which are stable against oxidative degradation caused by the formation of reactive peroxides. It is desired to provide polymer compositions for use in different applications or end-user products wherein the peroxide contents are not increased even upon storing for long periods of time, thereby providing end-user products with better stability and desired performance.

### SUMMARY OF THE INVENTION

One aspect of the present invention provides a peroxide stable polymer composition comprising: a mixture of (i) 95 wt. % to about 99.999 wt. % of polyvinylpyrrolidone/vinyl acetate copolymer (PVP/VA); and (ii) 0.001 wt. % to 5.0 wt. % of butylated hydroxy anisole (BHA).

In another aspect, the present invention provides a pharmaceutical composition comprising a peroxide stable polymer composition comprising a mixture of 95 wt. % to about 99.999 wt. % of polyvinylpyrrolidone/vinyl acetate copolymer (PVP/VA), and 0.001 wt. % to 5 wt. % of butylated hydroxy anisole (BHA); and at least one pharmaceutical active ingredient.

In yet another aspect, the present invention provides a process for preparing a peroxide stable polymer composition comprising the steps of: (i) preparing a feed mixture comprising 95 wt. % to about 99.999 wt. % of PVP/VA copolymer and 0.001 wt. % to 5 wt. % of BHA in an aqueous and/or organic solvent; and (ii) spray drying the feed mixture of process step (i) to form a free-flowing peroxide stable polymer composition comprising a mixture of PVP/VA copolymer and BHA.

Another aspect of the present invention provides a peroxide stable polymer composition consisting of: a mixture of (i) 95 wt. % to about 99.999 wt. % of polyvinylpyrrolidone/vinyl acetate copolymer (PVP/VA); and (ii) 0.001 wt. % to 5 wt. % of butylated hydroxy anisole (BHA).

### BRIEF DESCRIPTION OF THE DRAWINGS

Further embodiments of the present application can be understood with references to the appended figures.

FIG. 1 illustrates peroxide growth in the polymer compositions prepared in accordance with Example 1 (Ex. 1 with BHA) and Comparative Example 1 (Com. Ex. 1 with BHT).

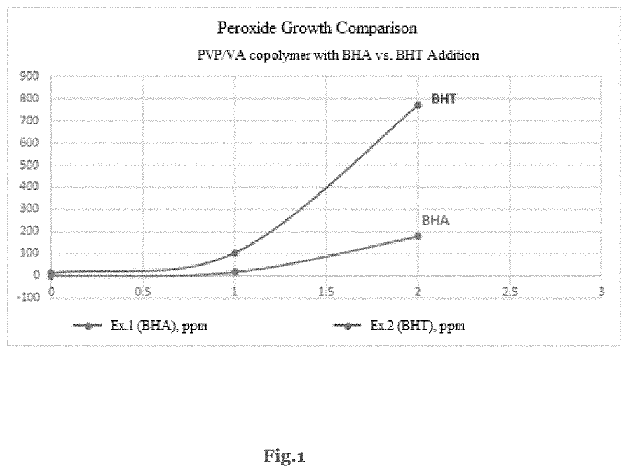

FIG. 2 illustrates Scanning Electron Microscopy (SEM) images of the polymer compositions of Example 1 (Ex. 1 with BHA) and Comparative Example 1 (Com. Ex. 1 with BHT).

FIG. 3 illustrates melting rheology profile of the polymer compositions of Comparative Example 1 (Ex. 1 with BHA) and Comparative Example 1 (Com. Ex. 1 with BHT).

FIG. 4 illustrates yellowness index of tablets prepared from polymer compositions of Example 1 (Ex. 1 with BHA) and Comparative Example 1 (Com. Ex. 1 with BHT).

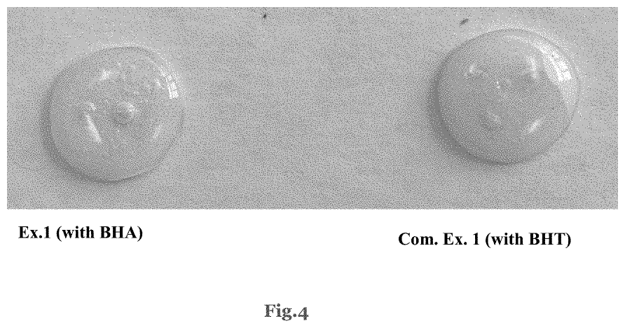

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

The term “about” can indicate a difference of 10 percent of the value specified. Numerical ranges as used herein are meant to include every number and subset of numbers enclosed within that range, whether particularly disclosed or not. Further, these numerical ranges should be construed as providing support for a claim directed to any number or subset of numbers in that range.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

As used herein, the words “preferred” or “preferably” and variants refer to embodiments of the application that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the application.

References herein to “one embodiment” or “one aspect” or “one version” or “one objective” of the application include one or more such embodiment, aspect, version or objective, unless the context clearly dictates otherwise.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

In one aspect, the present invention provides polymer compositions which are stable against the oxidative degradation caused by reactive peroxide.

In one of the embodiments, the present invention provides a peroxide stable polymer composition comprising a mixture of a polymer and at least one antioxidant.

Examples of polymers suitable for use in the peroxide stable composition of the present invention are: N-vinyl lactam polymers, polyethers, polyalkyleneimines, polyvinyl amines, polyvinyl formamide and partially hydrolyzed products thereof, polyimides and polyamides.

In one of the embodiments, the polymer is an N-vinyl lactam polymer. The N-vinyl lactam polymer can be a homopolymer or a copolymer of two or more of the monomers. In accordance with one of the embodiments, the N-vinyl lactam polymer is a copolymer of monomer (a), and monomer (b).

Examples of monomers (a) suitable for the purpose of the present invention are, for example:

N-vinyllactams, such as N-vinyl-2-pyrrolidone, N-vinylpiperidone, N-vinyl caprolactam, derivatives thereof substituted with C1- to C8-alkyl groups, such as 3-methyl-, 4-methyl- or 5-methyl-N-vinylpyrrolidone.

In one embodiment, suitable monomers (a) are N-vinyl-2pyrrolidone, 3-methyl-N-vinylpyrrolidone, 4-methyl-N-vinylpyrrolidone, 5-methyl-N-vinylpyrrolidone, N-vinylpiperidone and N-vinylcaprolactam

According to another embodiment, monomers (b) are vinyl acetate, and also the vinyl alcohol obtainable by hydrolysis after the polymerization, vinylamides such as vinylformamide, and also the vinylamine obtainable by hydrolysis after the polymerization, N-vinylimidazole, 1-vinyl-3-methylimidazolium chloride, 1-vinyl-3-methylimi dazolium sulfate, vinylmethylacetamide and derivatives thereof.

In accordance with one of the embodiments, the polymer is Poly(N-vinyl-2-pyrrolidone-co-vinyl acetate) (PVP/VA) copolymer. The PVP/VA copolymer possesses the general structure as shown in the below formula:

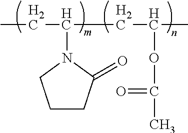

wherein “m” and “n” are independently an integer equal to or greater than 1. The PVP/VA copolymer can be a random, linear copolymer or a crosslinked copolymer. In accordance with one of the embodiments of the present invention, the PVP/VA is a linear random copolymer. The weight ratio of the N-vinyl-2-pyrrolidone monomers in PVP/VA copolymer of the present invention varies in the range of from about 50 wt. % to about 80 wt. %, or in the range of from about 70 wt. % to about 60 wt. %. Similarly, the weight percentage of the vinyl acetate monomers in the PVP/VA copolymer varies in the range of from about 20 wt. % to about 50 wt. % or in the range of from about 20 wt. % to about 40 wt. %. In accordance with one of the embodiments of the present invention, the N-vinyl-2-pyrrolidone and vinyl acetate monomers are present in an amount of 60 wt. % and 40 wt. %, respectively.

The PVP/VA copolymer can be prepared by the free-radical polymerization of N-vinyl-2-pyrrolidone and vinyl acetate monomers. The free radical polymerization can be carried out either as a solution polymerization or a precipitation polymerization in a suitable solvent such as water or mixture of water or suitable organic solvents. Examples of organic solvents suitable for the present invention include methanol, ethanol or isopropanol. The free radical polymerization process is a well-known process, and the PVP/VA copolymer of the present invention can be prepared by methods known to a person skilled in the related art.

The PVP/VA copolymer containing the specific ratio of about 60% N-vinyl-2-pyrrolidone (PVP) and about 40% vinyl acetate (VA) is also known as copovidone. Commercially manufactured PVP/VA copolymers include, but are not limited to, Plasdone® S-630, PVP/VA, by Ashland Specialty Ingredients and Kollidon®, PVP/VA by BASF.

The PVP/VA copolymer in accordance the present invention has a K-value in the range of from about 10 to about 150 or in the range(s) of from about 15 to about 30, about 30 to about 60, about 60 to about 90, about 90 to about 120, or about 120 to about 150. In accordance with one of the embodiments of the present invention, the K-value of the PVP/VA copolymer ranges from about 25 to about 32. The K-value of the PVP/VA copolymer is a function of the average molecular weight, the degree of polymerization and the intrinsic viscosity. The K-value is derived from viscosity measurement and is calculated according to Fikentcher's formula. The weight average molecular weight of the PVP/VA copolymer of the present invention varies in the range of from about 20,000 to 40,000 Daltons or in the range of from about 24,000 to 30,000 Daltons.

The amount of PVP/VA copolymer in the peroxide stable polymer composition of the present invention is in the range of from about 95 wt. % to about 99.999 wt. %, or in the range of about 95 wt. % to about 96 wt. %, about 96 wt. % to about 97 wt. %, about 97 wt. % to about 98 wt. %, about 98 wt. % to about 99 wt. %, or about 99 wt. % to about 99.99 wt. %.

The peroxide stable polymer composition of the present invention further comprises at least one antioxidant. The term “antioxidant” in the context of the present invention refers to a substance, preferably ‘organic substance’ which when used in the polymers of the present invention inhibits oxidative degradation thereof under the influence of heat and/or air. Examples of antioxidants suitable for use in the present invention include, but are not limited to, butylated hydroxy anisole (BHA) or butylated hydroxy toluene (BHT). In accordance with one of the embodiments of the present invention, the antioxidant is butylated hydroxy anisole (BHA). The amount of antioxidant used in the peroxide stable polymer composition of the present invention can vary from about 0.001 wt. % to about 5.0 wt. %, or in the range of from about 0.1 to about 4.0 wt. %, from about 1.0 to about 2.0 wt. %, from about 2.0 to about 3.0 wt. %, from about 3.0 to about 4.0 wt. %. In accordance with one of the embodiments of the present invention, the antioxidant is present in an amount of from about 0.5 wt. % to about 1.5 wt. %.

The peroxide stable polymer composition according to the present invention can be prepared by means known in the art wherein the antioxidant can be added before, during or after the polymerization. In accordance with one of the embodiments of the present invention, the antioxidant is added after the polymerization. The addition of antioxidant to the polymers of the present invention can be carried out by means known in the related art by using suitable solvent medium such as water, organic solvents or mixtures thereof. Examples of the organic solvents are methanol, ethanol, isopropanol or mixtures thereof. In one of the embodiments, the polymer, PVP/VA copolymer is mixed with the solvent under continuous stirring at room temperature of about 25° C. to obtain a polymer solution. The polymer solution is then mixed with the antioxidant under continuous stirring to obtain the peroxide stable composition of the present invention. The peroxide stable composition can be converted to solid form by drying. Drying methods are known to the person skilled in the art. The drying of the peroxide stable polymer composition of the present invention can take place, for example, by spray-drying, drum-drying or any other warm-air drying or contact-heat drying methods. In one of the embodiments of the present invention, the peroxide stable polymer composition is dried by spray-drying.

The peroxide stable polymer composition of the present invention demonstrates excellent stability upon storage against peroxide formation. The stability of the peroxide stable polymer composition of the present invention is determined by measuring the peroxide content present therein at different time intervals. The peroxide content in parts per million (ppm) level are typically measured by using a suitable method that is readily known and available to a person skilled in the pertinent art. In accordance with one of the embodiments of the present invention, the peroxide content range for the mixture of PVP/VA copolymer and anti-oxidant is not more than 180 ppm after the storage of 1 to 3 weeks at a temperature of 60° C. in HDPE (High density polyethylene) bottles, and in another embodiment, the peroxide content range for the mixture of PVP/VA copolymer and anti-oxidant is not more than 120 ppm after the storage of 1 to 3 weeks at a temperature of 60° C. in HDPE bottles. Further, it is contemplated that the peroxide content for the mixture of PVP/VA copolymer and anti-oxidant is not more than 180 ppm after the storage of 1 to 3 weeks at a temperature of 60° C. in any type of bottles other HDPE including but not limited to LDPE, borosil, glass, amber, plastic, PET, etc.

In another embodiment, the present invention provides a peroxide stable composition consisting of a mixture of (i) about 95 wt. % to about 99.999 wt. % of polyvinylpyrrolidone/vinyl acetate (PVP/VA) copolymer, and (ii) about 0.001 wt. % to about 5.0 wt. % butylated hydroxy anisole.

In addition to the stabilization against peroxide formation, the peroxide stable composition of the present invention is also advantageous in terms of color stability. The color and odor of peroxide stabilized polymer composition barely changes over due course due to the presence of no or low peroxide content.

Accordingly, the peroxide stable polymer composition of the present invention can be advantageously used in a variety of applications such as pharmaceuticals, cosmetics, agricultural chemicals, food technology, animal health, animal feed, beverage technology, photosensitive electron materials, and adhesion providing agents. According to one of the embodiments of the present invention, the peroxide stable polymer composition is used in pharmaceutical compositions.

In another aspect, the present invention provides a pharmaceutical composition comprising the peroxide stable polymer composition of the present invention. The peroxide stabilized polymer composition can be used either as an active ingredient or as an excipient. According to one of the embodiments of the present invention, the pharmaceutical composition comprises peroxide stabilized polymer as an excipient and at least one active pharmaceutical ingredient (API). The API includes, but is not limited to, at least one ingredient selected from the group consisting of antibiotics, anti-inflammatory agents, antifungal agents, anti-infectives, immunosuppressants, anti-depressants, anti-cancer agents, anti-tubercular agents, cardiovascular agents, gastrointestinal agents, anti-viral agents, anti-psychotic agents, anti-histamines, anti-diabetic agents, cholesterol lowering agents, immune modulators, anti-epileptic agents, analgesic agents, anti-psoriatic agents, anti-pyretics, anti-malarial agents, antiseptics, mucolytics, decongestants, sedatives, anti-coagulants, diuretics, cholinergics, and dopaminergics.

Optionally, additional excipient(s) can also be used. Examples of non-limiting additional excipients suitable for the pharmaceutical composition of the present invention include pharmaceutical lubricants, disintegrants, binders, humectants, glidants, fillers, surfactants or mixtures thereof.

The pharmaceutical composition according to one embodiment of the present invention can be formulated into solid dosage forms selected from the group consisting of soft gelatin capsule, tablets, capsules, pill, particulates, granules, powder, disc, caplets, sachets, and suspension. The solid dosage form according to one of the embodiments of the present invention is particularly suitable for oral administration. Methods for preparing various dosage forms are known in the related art. Accordingly the pharmaceutical composition of the present invention can be formulated into solid dosage forms by conventional methods.

The present invention is further illustrated by the following non-limiting examples. These examples are for the illustration purpose only and not to be construed as limiting the scope of the present invention.

### EXAMPLES

Testing Methods Details:

Determination of Peroxide Contents in the Polymer Compositions:

The peroxide content in the polymer composition of the present invention was calculated based upon the European Pharmacopoeia Method.

For the peroxide growth experiment, each sample was tested one day after spray drying for an initial peroxide value (t=0). Then, each sample was aged at 60° C. in an oven and tested for peroxide concentration at 1, 2, and 3 weeks. The values for each sample were duly evaluated to understand the peroxide growth comparison.

K-Value Determination:

The K-value of PVP/VA copolymer of the present invention in either an ethanol or aqueous solution is defined by the Fikentscher equation:

$$\frac{log {η}_{rel}}{C} = \frac{75 {K}_{O}^{2} + {K}_{O}}{1 + 1.5 {K}_{O} C} + {K}_{O}$$

when K=1000 Ko, C=concentration in g/100 ml, and ηrel=relative viscosity.

The relative viscosity of the PVP/VA copolymer solution with the specified concentration (C) is determined. The K-value can calculated according to the following equation

$$K = \frac{\sqrt{300 c log {η}_{r e l} + {( c + 1.5 c log {η}_{r e l} )}^{2}} + 1.5 c log {η}_{r e l} - c}{0.15 c + 0 .003 {c}^{2}}$$

In this method, K-value at 1.00% was determined from the K-value and relative viscosity correlation table in the end of this method.

Mol. wt. Determination: To determine weight average and number average molecular weight of the polymer, and polydispersity index, a size exclusion chromatography method, or SEC-RI, was used in which the molecular weight values are determined relative to a specific polymer standard.

### Example 1 (Ex. 1): Preparation of Present Polymer Composition

1164 gm of Plasdone S630 was diluted to 12% solid by adding 2043 g of DI water in a reaction vessel. Plasdone S630 commercial grade material was collected after carbon treatment and before spray drying. The % Solids for the carbon treated material was 33.07%. The aqueous solution of Plasdone S630 thus obtained was mixed with 200 ppm of butylated hydroxy anisole (BHA) diluted with 200 ppm of isopropyl alcohol under continuous stirring to obtain a polymer composition. The polymer composition was mixed well under continuous stirring and thereafter dried using spray drying technique. The spray drying was carried out using Yamato spray dryer (model# Pulvis GB22). The spray dryer was preheated to inlet temperature of 190° C. and outlet temperature of 90° C. The polymer composition was fed into a drying chamber using pump at the rate of 250 mol solution in 40 minutes. The feed rate was adjusted so that the outlet temperature does not fall below 78° C. The polymer compositing was obtained in white free flowing powder form which was collected in the receiver and transferred to jar. The peroxide content of the polymer compositions was determined immediately after preparing the polymer composition in dried free flowing powder form and, also after storage for 1, 2 and 3 weeks consecutively.

### Comparative Example 1 (Com. Ex. 1)

Another set of experiments was carried out in the same manner as described in Example 1, except butylated hydroxy toluene (BHT) was used instead of BHA as the antioxidant. The determination of peroxide content was also carried out similar to example-1. The peroxide contents of the polymer compositions of Ex. 1 and Com. Ex. 1 are illustrated in Table-1.

TABLE 1
Peroxide levels (ppm) in Example 1 and Comparative Example 1

| Time (Weeks) in | Peroxide Level, ppm | Peroxide Level, ppm |
|---|---|---|
| Oven at 60° C. | (Ex. 1) with BHA | (Com. Ex. 1) with BHT |
| 0 | 0 | 14 |
| 1 | 18 | 105 |
| 2 | 178 | 771 |
| 3 | 239 | 842 |
| Water Content | 3.50% | 3.56% |
| Original | | |

As evident from the data provided in Table 1, BHA antioxidant showed superior peroxide inhibition (lower peroxide concentration) as compared to BHT antioxidant. BHA gave lower peroxide concentrations, both at time zero and with aging as well.

The polymer compositions of Ex. 1 and Comp. Ex. 1 were further analyzed with respect to density particle size distribution, flowability and thermal properties. These properties were illustrated in Table 2.

TABLE 2
PVA/VA copolymer properties of Example
1 and Comparative Example 1

| Properties (Unit) | | Ex. 1 | | Com. Ex. 1 | |
|---|---|---|---|---|---|
| Density | Ture | 1.2333 | | 1.2279 | |
| (g/cm³) | Bulk | 0.0948 | | 0.1003 | |
| | Tap | 0.1523 | | 0.1588 | |
| Particle Size | D10 | 2.48 | | 2.15 | |
| and | D50 | 8.51 | | 8.03 | |
| Distribution | D90 | 21 | | 20.2 | |
| (μm) | Average | 11.4 | | 10.8 | |
| Flowability | Flow function | 4.5 | (cohesive) | 5.7 | (cohesive) |
| | (Flow | | | | |
| | category) | | | | |
| | Carr Index (%) | 37.8 | (very poor) | 36.8 | (very poor) |

| | | | |
|---|---|---|---|
| Thermal | Glass transition | 111.5 | 111.5 |
| Properties | Degradation | 313 | 314 |
| (° C.) | under Air | | |
| | Degradation | 316 | 316 |
| | under N2 | | |

| | | |
|---|---|---|
| Water Content TGA (%) | 3.9 | 4.3 |

From Table 2, it is evident that both the polymer compositions have comparable true, bulk and tap density. Both the polymer compositions also displayed similar particle size and distribution, which was significantly smaller than commercial S630 due to lab scale spray dryer used. Both the polymer compositions showed poor flowability, however, the polymer composition of Com. Ex. 1 (BHT) has slightly better flowability and the flowability based on FF is cohesive. Both the polymer compositions showed identical glass transition temperature and thermal stabilities under both air and N₂. The antioxidants did not have any impact on thermal properties. Further, both the polymer compositions showed similar water content.

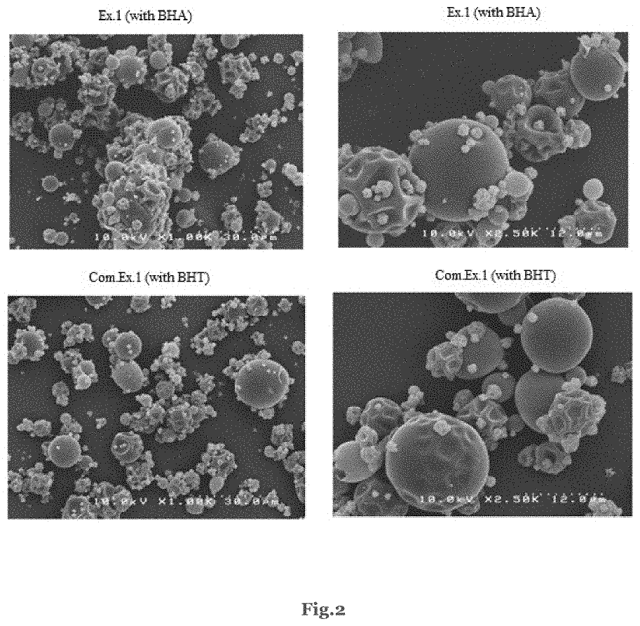

The surface morphology of both the polymer compositions was also imaged by using scanning electron microscope (SEM). The SEM images are provided in FIG. 2. The SEM images showed that both polymer compositions have typical spray-dried materials morphology, which is spherical or semi-spherical. In addition, the images proved that both antioxidants have non-significant impact to the spray dry process, manifested by similar morphology and particle size and distribution of the products.

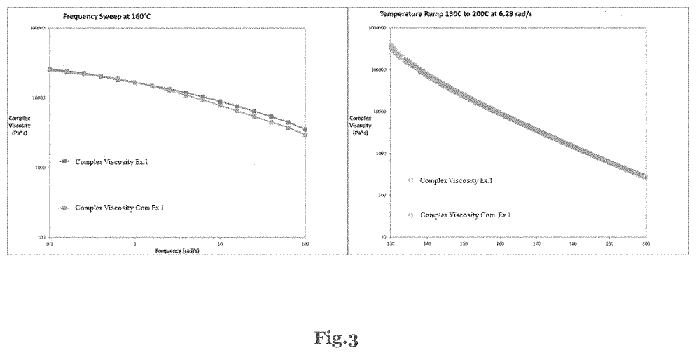

Further, melting rheology of the both the polymer compositions was monitored by using a AR 2000 rheometer. As evident from FIG. 3, both the polymer compositions displayed nearly identical rheological responses under frequency and temperature sweeps, which indicated that the antioxidant has no-impact on the rheological properties of PVP/VA copolymer. Therefore, both the polymer compositions should have virtually the same thermal processability, i.e. extrudability in a hot melt extrusion (HME) process.

The color stability of the polymer compositions of Ex. 1 and Com. Ex. 1 was also evaluated. For this, both the polymer compositions were formulated in the form of tablets using conventional means. The yellowness index of tablets (stressed at 180° C. for 1 hour) derived from both the polymer compositions was read. The tablet derived from the polymer compositions of Ex. 1 (with BHA) showed better performance (less yellow and smaller yellowness index value) than the tablet derived from the polymer composition of Com. Ex. 1 (with BHT), which was consistent with the peroxide data showed in Table 1 showing that BHA unexpectedly controls peroxide growth compared to BHT.

All of the articles and/or methods disclosed herein can be made and executed without undue experimentation in light of the present disclosure. While the articles and methods of the disclosed and/or claimed inventive concept(s) have been described in terms of particular aspects, it will be apparent to those of ordinary skill in the art that variations may be applied to the articles and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

What is claimed is:

1. A peroxide stable polymer composition comprising:
a mixture of (i) from 95 wt. % to about 99.999 wt. % of polyvinylpyrrolidone/vinyl acetate (PVP/VA) copolymer; and (ii) from 0.001 wt. % to 5.0 wt. % of butylated hydroxy anisole (BHA).

2. The peroxide stable polymer composition according to claim 1, wherein the BHA is present in an amount of from 0.1 to 4.0 wt. %.

3. The peroxide stable polymer composition according to claim 1, wherein the BHA is present in an amount of from 0.5 to 1.5 wt. %.

4. The peroxide stable polymer composition according to claim 1, wherein the PVP/VA copolymer is a linear random copolymer having PVP monomers in an amount of from 50 wt. % to 80 wt. %, and VA monomers in an amount of from 20 wt. % to 50 wt. %.

5. The peroxide stable polymer composition according to claim 1, wherein the weight ratio of the PVP and VA monomer is 60:40.

6. The peroxide stable polymer composition according to claim 1, wherein the PVP/VA copolymer has a weight average molecular weight ranging from 20,000 to 40,000.

7. The peroxide stable polymer composition according to claim 6, wherein the weight average molecular weight of the PVP/VA copolymer is in the range of from 24,000 to 30,000.

8. The peroxide stable polymer composition according to claim 1, wherein the PVP/VA copolymer has a K value ranging from 10 to 150.

9. The peroxide stable polymer composition according to claim 8, wherein the K value of the PVP/VA copolymer ranges from 25 to 32.

10. The peroxide stable polymer composition according to claim 1, wherein the polymer composition has peroxide content varying in the range of from 0 to 180 ppm, based on solid content of the PVP/VA copolymer.

11. The peroxide stable polymer composition according to claim 10, wherein the peroxide content varies in the range of from 0 to 120 ppm based on solid content of the PVP/VA copolymer.

12. A pharmaceutical composition comprising:
(i) a peroxide stable polymer composition comprising a mixture of from 95 wt. % to 99.999 wt. % of polyvinylpyrrolidone/vinyl acetate copolymer (PVP/VA), and from 0.001 wt. % to 5 wt. % of butylated hydroxy anisole (BHA); and
(ii) at least one active pharmaceutical ingredient (API).

13. The pharmaceutical composition according to claim 12, wherein the composition is formulated in a solid dosage form selected from the group consisting of soft gelatin capsule, tablets, capsules, pellets, particulates, granules, powder, disc, caplets or sachets.

14. The pharmaceutical composition according to claim 12, wherein the active ingredient is selected from the group comprising antibiotics, anti-inflammatory agents, antifungal agents, anti-infectives, immunosuppressants, anti-depressants, anti-cancer agents, anti-tubercular agents, cardiovascular agents, gastrointestinal agents, anti-viral agents, anti-psychotic agents, anti-histamines, anti-diabetic agents, cholesterol lowering agents, immune modulators, anti-epileptic agents, analgesic agents, anti-psoriatic agents, anti-pyretics, anti-malarial agents, antiseptics, mucolytics, decongestants, sedatives, anti-coagulants, diuretics, cholinergics, dopaminergics, and mixtures thereof.

15. A process for preparing a peroxide stable polymer composition comprising the steps of:
(i) preparing a feed mixture comprising from 95 wt. % to 99.999 wt. % of PVP/VA copolymer and from 0.001 wt. % to 5 wt. % of BHA in an aqueous and/or organic solvent; and
(ii) spray drying the feed mixture of process step (i) to form a free-flowing peroxide stable polymer composition comprising a mixture of PVP/VA copolymer and BHA.

16. The process according to claim 15, wherein the solvent includes at least one solvent selected from the group consisting of isopropanol, ethanol, water, dichloromethane, chloroform, ethyl acetate, ethyl methyl ketone, methanol, acetone, and mixtures thereof.

17. The process according to claim 16, wherein the solvent is selected from the group consisting of water, isopropanol or a mixture thereof.

18. A peroxide stable polymer composition consisting of:
a mixture of (i) from 95 wt. % to 99.999 wt. % of polyvinylpyrrolidone/vinyl acetate copolymer (PVP/VA); and (ii) 0.001 wt. % to 5 wt. % of butylated hydroxy anisole (BHA).
